(19) United States
(12) Patent Application Publication
(10) Pub. No.: US 2007/0282183 A1
(43) Pub. Date: Dec. 6, 2007

(54) Method and device for the identification of at least two substances of content of a body fluid

(75) Inventors: Bernd Scholler, Karlsruhe (DE); Michael Bernstein, San Ramon, CA (US); Karl-Andreas Feldhahn, Hamburg (DE); Klaus Forstner, Asperg (DE)

Correspondence Address: Friedrich Kueffner, 317 Madison Avenue, Suite 910, New York, NY, 10017, US

(73) Assignee: Weinmann Gerate fur Medizin GmbH & Co. KG

(21) Appl. No.: 11/787,104

(22) Filed: Apr. 12, 2007

(30) Foreign Application Priority Data
Apr. 12, 2006 (DE) 10 2006 018 043.7

(51) Int. Cl. A61B 5/1455

(52) U.S. Cl. 600/328

(57) ABSTRACT

A method and a device for the identification of at least two substances of content of a body fluid, wherein adjacent to a body tissue containing the body fluid, are arranged at least one radiation source and a photo receiver. The radiation source generates radiation of two different wavelengths. The radiation is directed onto the body tissue and the photo receiver receives radiation reflected by the body tissue and/or reduced through the body tissue. At least at times the radiation of a third wavelength is directed onto the body tissue for the identification of a hemoglobin derivate.

### BACKGROUND OF THE INVENTION

1. Field of the Invention

The present invention relates to a method and a device for the identification of at least one substance of content of a body fluid, wherein adjacent to a body tissue containing the body fluid at least one radiation source as well as a photo receiver are arranged , wherein the radiation source generates radiation of at least two different wavelengths and wherein the radiation is directed onto the body tissue and the photo receiver receives radiation that is reflected by the body tissue and/or is reduced through the body tissue.

2. Description of the Related Art

It is known in the art to conduct a radiography and/or a backscatter with a desired wavelength in a definable intensity on body tissue with wavelength-dependent absorption coefficients and/or with strong light scatter, in which an adjustment desired and/or defined by the user can be effected manually or through automatic regulation.

The long established pulsoximetry allows for a non-invasive measurement of the oxygen saturation of the arterial blood. For this, for example, the light of two different wavelengths, for example, 660 nm and 905 nm is guided through a finger, which is partially absorbed by the blood pulsating through the tissue. The degree of absorption is defined through an analysis of the portion of the light exiting on the other side of the radiographed tissue, which allows an immediate conclusion as to the oxygen saturation of the pulsating and thus arterial blood.

The pulse spectroscopy expands the non-invasive diagnostic, among other things, by the following blood parameters: concentration of hemoglobin, absolute oxygen saturation of the blood, carbon monoxide concentration, concentration of methemoglobin, concentration of bile pigment. When conducting a pulse spectroscopy, like in a pulsoximetry, also wavelengths of, for example, 660 nm and 905 nm are used, however, further wavelengths are necessary. The principles of the pulse spectroscopy are illustrated in the following patent documents: DE 103 21 338 A1, DE 102 13 692 A1 and DE 10 2005 020 022 A1.

In media with wavelength-dependent absorption, the intensity of the radiation changes with the distance and the spectral composition. This is also true for the scattering of the radiation, because it weakens the radiation due to the size and the number of the dispersion centers and it also changes the radiation spectrally with distance. Therefore, radiation sources are needed that can optimally compensate these changes in order to facilitate an evaluation of the reflected dispersed portion and/or of the portion after a radiography.

Such changes of radiation are caused, for example, by a wavelength dependent absorption of the substances of content of a body fluid like, for example, hemoglobin, glucose, bile pigment, and water which can be described by approximation through the Beer Lambert law.

The absorption of radiation of a defined wavelength can be quickly estimated with the help of the absorption coefficient. The absorption coefficient of water shows a strong wavelength dependency. Water molecules show a strong absorption band at approximately 1450 nm.

Hemoglobin, for example, has two transmission bands in the red and in the blue-green zone.

### SUMMARY OF THE INVENTION

It is the object of the present invention to develop a method and a device which ensure, with one configuration, a plurality of measurements of blood substance content, wherein a radiation characteristic is changeable according to necessity and has a minimum power requirement, wherein at least one identification of two substances of content of a body fluid is guaranteed.

In accordance with the present invention, adjacent to a body tissue containing the body fluid at least one radiation source and a photo receiver are arranged, wherein the radiation source generates radiation of at least two different wavelengths, directing the radiation onto the body tissue, the photo receiver receiving radiation that is reflected by the body tissue and/or receiving radiation that is reduced through the body tissue, further comprising at least temporarily directing radiation of a third wavelength onto the body tissue for the identification of a hemoglobin derivate.

In accordance with another embodiment, adjacent to a body tissue containing the body fluid at least one radiation source and a photo receiver are arranged, wherein radiation of two different wavelengths is generated by the radiation source, wherein the radiation is directed onto the body tissue and the photo receiver receives radiation reflected by the body tissue or reduced through the body tissue, wherein through control of a user selection and/or through automatic control at least temporarily radiation of a third wavelength is directed onto the body tissue for the identification of the hemoglobin concentration, further comprising directing additionally at least temporarily radiation of a fourth wavelength onto the body tissue for. the identification of the carbon monoxide concentration of the hemoglobin.

In accordance with another embodiment, adjacent to a body tissue containing the body fluid at least one radiation source and a photo receiver are arranged, wherein radiation of two different wavelengths is generated by the radiation source, wherein the radiation is directed onto the body tissue and the photo receiver receives radiation reflected by the body tissue or reduced through the body tissue, wherein through control of a user selection and/or through automatic control at least temporarily radiation of a third wavelength at which water absorbs more strongly than hemoglobin is directed onto the body tissue for the identification of the hemoglobin concentration, further comprising directing additionally at least temporarily radiation of a fourth wavelength in the range of 600 nm to 710 nm onto the body tissue for the identification of a carbon monoxide concentration of the hemoglobin.

The device for the identification of at least two substances of content of a body fluid includes at least one radiation source for the generation of two wavelengths, wherein the radiation source and the photo receiver are equipped with a clamping arrangement for positioning them in the area of a body tissue containing a body fluid, and wherein the radiation source emits at least temporarily radiation of a third wavelength.

The source of electromagnetic radiation is, for example, one or several laser diodes and/or one or several white light sources and/or one or several LED.

The object of the invention is further solved by using different light emitting diodes (LED) with equal and/or different configuration. The use of light emitting diodes guarantees, on the one hand, a long life span and low energy consumption, so that two of the above mentioned demands would already be satisfied. The invention is distinguished through further characteristics that make as much use of the good activation of the LED as well as its emission characteristics and its different radiation characteristics.

The method and device according to the invention provides a solution with which a non-invasive identification of at least one substance of content of a body fluid chosen from the group of pulse frequency, ph-value, concentration of hemoglobin (cHb), oxyhemoglobin (HbO2), desoxygenized hemoglobin (HbDe), carboxyhemoglobin (HbCO), methemoglobin (cMetHb), sulfhemoglobin (HbSulf), bile pigment, glucose, bile pigments, SaO2, SaCO, SpO2, CaO2, SpCO, is made possible. Further, a non-invasive identification of several substances of content of a body fluid is possible.

For the realization, an important feature of the light emitting diodes is their activation through their non-linear current-tension-characteristic curve according to the Shockley equation.
I=Iexp(Uf/nkT)

I: flow stream; UF: flow tension; I: saturation flow; k: Boltzmann constant; T: absolute temperature, n: constant (with a value between 1 and 2).

Since the number of emitted photons over a great flow area is directly proportional to the flow stream, LEDs are easily controlled regarding their light intensity over several ranges through a small change in the flow tension.

Theoretically, changes in the flow tension of up to 150 mV are possible. This would cause a change of flow tension by factor 10 and a change of luminosity also by 10.

GaAIAs/GaAs (red and infrared): 1.2 to 1.8 V

InGaAIP (red and orange): 2.2 V

GaAsP/GaP (yellow): 2.1 V

GaP/GaP (green): 2.1 V

InGaN (blue and white): 3.3 to 4 V Silicon diode: 0.7 V

The power input varies from one model to another between 2 mA, 20 mA (for example 5-mm-LED) up to approximately 700 mA or more in LED for purposes of illumination. The conducting state voltage (Uf) hereby ranges from approximately 1.5 V (infrared-LED) to approximately 4 V (InGaN-LED: green, blue UV).

This creates the possibility, when using different LEDs, to quickly manage and purposefully change an additive complement of the luminosity/light intensity through targeted regulation of one kind of LED.

Thus, it is possible, in selective absorption, as it can occur in water or in blood (through hemoglobin), and by using different LEDs to control one kind of LED through its current in such a way that different tissue thicknesses, skin pigmentations and other factors can be considered in such a manner that a photo receiver always receives a defined portion of scattered radiation and/or reduced radiation for evaluation.

A further characteristic of LED is its varied irradiation characteristic which can show aperture angles from 2° to 45°; in addition, almost cosine-like irradiation is possible.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a schematic illustration of an LED arrangement;

FIG. 2 is a further schematic illustration of an LED arrangement;

FIG. 3 is schematic illustration of a finger clip sensor;

FIG. 4 shows a typical absorption process in a measuring of blood and water;

FIG. 5 shows absorption spectrums of functional and dysfunctional hemoglobin derivates; and

FIG. 6 shows a typical process of the absorbancy coefficient for various hemoglobin derivates.

### DETAILED DESCRIPTION OF THE INVENTION

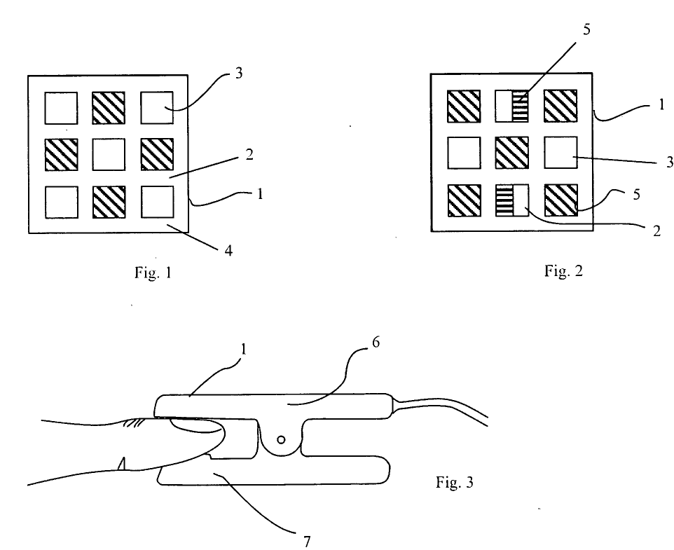

The LED arrangement 1 as shown in FIG. 1 and FIG. 2, respectively, includes numerous LED which are mounted on a collective carrier 4, for example, a circuit board with adequate conduct structures (not shown) for the electrical supply and the activation of the LED. Alternatively, the carrier can also be designed as a finger clip sensor.

FIG. 2, in addition, shows an LED 5 which emits two wavelengths.

FIG. 3 shows a finger clip sensor 6 with integrated LED arrangement 1 and photo receiver 7.

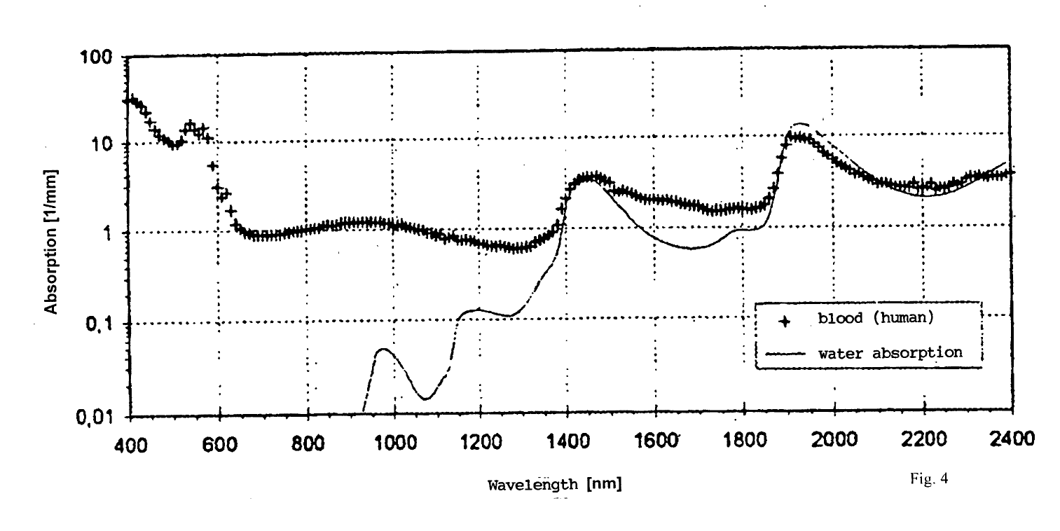

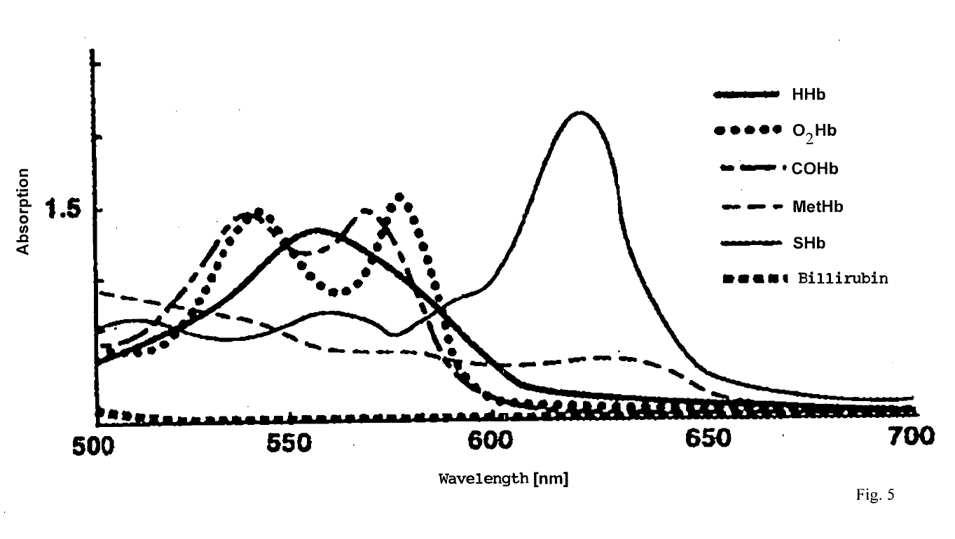

FIG. 4 shows a typical absorption process for the measuring of blood and water. One recognizes absorption maxima for water in the range of wavelengths of 950 nm, 1200 nm, 1450 nm, 1900 nm and 2400 nm. One recognizes absorption maxima for blood in the range of wavelengths of 550 nm, 910 nm, 1450 nm and 1900 nm.

FIG. 5 shows a typical absorption process for the measuring of the oxygen saturation in blood. An absorption intensity is assigned in dependence on the respective wavelength. A first minimum is encountered at a wavelength of approximately 600 nanometer. Starting at approximately 680 nanometer, the progression approaches asymptotically the zero line.

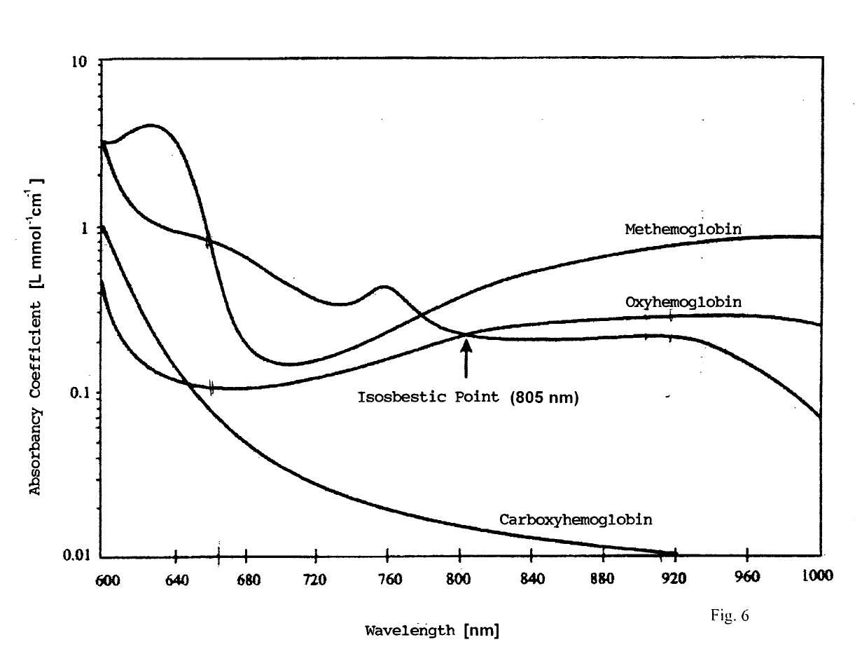

FIG. 6 shows a typical process of the typical course of the absorbancy coefficients for various hemoglobin derivates. At 805 nm is the isosbestic point, here the absorbancy of oxyhemoglobin is equal to the absorbancy of desoxyhemoglobin.

The LED are respectively connectable with an LED control device. The LED control device regulates the power and/or voltage supply of each individual LED.

The LED are covered with a coating (not shown).

The LED have at least three different emission wavelengths. According to the invention, there are at least three LED for every emission wavelength in the area of the LED device. One of the two LED for one emission wavelength is the main LED, the at least one further LED of the same emission wavelength serves as auxiliary LED.

With the help of these auxiliary LED 3 those spectral components are added to the over-all spectrum which are missing in the emission spectrum of the active main LED 4 for the identification of the desired substance of content.

Preferably, the main and/or auxiliary LED are configured in such a way that they can emit alternatively and/or complementary the following wavelengths selected from the group:

150 nm±15%, 400 nm±15%, 460 nm±15%, 480 nm±15%, 520 nm±15%, 550 nm±15%, 560 nm±15%, 606 nm±15%, 617 nm±15%, 620±15%, 630 nm±15%, 650 nm±15%, 660 nm±15%, 705 nm±15 %, 710 nm±15%, 720 nm±10%, 805 nm±15%, 810 nm±15%, 880 nm±15%, 890 nm, 905 nm±15%, 910 nm±15%, 950 nm±15%, 980 nm±15%, 1000 nm±15%, 1030 nm±15%, 1050 nm±15%, 1100 nm±15%, 1200 nm±15%, 1310 nm±15%, 1380 nm±15%, 1450 nm±15%, 1600 nm±15%, 1650 nm±15%, 1670 nm±15%, 1730 nm±15%, 1800 nm±15%, 2100 nm±15%, 2250 nm±15%, 2500 nm±15%, 2800 nm±15%

TABLE 1

| Wavelength (nm) | LED material |
|---|---|
| 940 | GaAIAs/GaAs |
| 880 | GaAIAs/GaAs |
| 850 | GaAIAs/GaAs |
| 660 | GaAIAs/GaAs |
| 635 | GaAsP/GaP |
| 633 | InGaAIP |
| 620 | InGaAIP |
| 612 | InGaAIP |
| 605 | GaAsP/GaP |
| 595 | InGaAIP |
| 592 | InGaAIP |
| 585 | GaAsP/GaP |
| 574 | InGaAIP |
| 570 | InGaAIP |
| 565 | GaP/GaP |
| 560 | InGaAIP |
| 555 | GaP/GaP |
| 525 | SiC/GaN |
| 505 | SiC/GaN |
| 470 | SiC/GaN |
| 430 | SiC/GaN |
| 660/910 | AIGaAs |
| 660/850 | |
| 660/940 | |
| 635/760 | |
| 565/660 | |
| 760/940 | |

Table 1 shows an exemplified list of suitable LED which can be used in accordance with the invention.

According to the invention, it is also considered to use two-wavelengths emitting LED. Preferably, there are used two-wavelengths emitting LED where the intensities of each of them can be controlled independently.

For example, for the identification of the hemoglobin concentration at least two LED emit in the range of, for example, 1450 nm±15% and 660 nm±15% and 905 nm±15%.

Complementary, a further wavelength in the range of 605 nm can be additionally activated through a selected medium, for example, for the identification of the carbon monoxide parts.

It is also taken into consideration that the auxiliary LED emits at a wavelength range of ±15% of the wavelength of the main LED. According to the invention, the auxiliary LED is preferably arranged at a distance of at least one mm from the main LED. Through co-activation of the auxiliary LED, the leftover intensity of the radiation after passing through the tissue is again sufficient for an evaluation.

In another embodiment, the radiation source emits in the range of, for example, 660 nm±15% and in the infrared range of 890 nm±15% or 910 nm±15% for the identification of SpO2.

For the identification of the hemoglobin concentration, at least temporarily one further wavelength is selected manually and/or automatically that has a high water absorption, for example, in the area chosen from the group 1200 nm±15%, 1380 nm±15%, 1450 nm±15%, 1900 nm±15%, 2400 nm±15%.

For the identification of the carboxyhemoglobin concentration, at least temporarily one further wavelength is selected manually and/or automatically, for example, in the area chosen from the group 605 nm±15%, 606 nm±15% and 630 nm +15%.

According to the invention its is provided to hold out redundancies from other LED that emit at a same wavelength range in order to compensate a failing LED through another LED of the same wavelength and/or in order to increase the intensity at one wavelength. For example, 8 or 9 LED are used.

A further embodiment shows the procedure for the identification of at least two substances of content of a body fluid, wherein adjacent to a body tissue containing the body fluid there is arranged at least one radiation source as well as a photo receiver, and wherein the radiation source generates radiation of at least three different wavelengths which are directed onto the body tissue, and wherein the photo receiver receives radiation reflected and/or reduced by the body tissue in such a way that at least for one wavelength defined activation and defined deactivation periods are provided.

The activation periods and the deactivation periods are realized during the running of the device according to the invention and refer, for example, to the activation of the radiation sources, preferably LED. The activation periods thus describe phases during which certain wavelengths are emitted, and the deactivation periods thus describe phases during which certain wavelengths are not emitted.

According to the invention, the activation periods and the deactivation periods are carried out, for example, by turning on or turning off the radiation source.

For the identification of, for example, the hemoglobin concentration, in one activation period radiation of at least three different wavelengths is directed onto the body tissue, wherein at least one of the three wavelengths, in the present case a wavelength at which water has a high absorption, only temporarily all n cycles are present in the activation period. This is a preferred implementation since the hemoglobin concentration is relatively constant and it changes rather slowly. Therefore, a continuous identification of the hemoglobin concentration does not make sense most of the time.

According to the invention, the deactivation periods help save energy. This is particularly advantageous in portable devices to prolong its operating time.

The deactivation periods for the interruption of activation periods can be carried out cyclically, wherein for the cycles N=2 is valid indefinitely.

According to the invention, at least one wavelength has a different activation period than the other two wavelengths.

According to the invention, for at least one wavelength an activation period and a deactivation period follow each other. Wherein the activation period and the deactivation period alternate and for the cycles N=2 is indefinitely valid.

In the identification of the hemoglobin concentration, for example, the deactivation period lasts for at least one wavelength in the time frame of three seconds up to one hour, while at least two further wavelengths are at least temporarily included in an activation period. The oxygen saturation is defined by the two wavelengths that are in the activation mode. Sporadically, through the activation of a further wavelength with a high water absorption, the identification of the hemoglobin concentration is made possible.

According to the invention, it is also considered that for at least one wavelength the activation period can last up to one hour or longer. In the identification of carboxyhemoglobin (SaCO) a long term monitoring of SaCO is expedient. Herein, one of the wavelengths, at which carboxyhemoglobin has a high absorption, is kept in the activation period for the length of the identification.

Parallel heret, at least two further wavelengths are at least temporarily present in the activation period for the identification of the oxygen saturation and the pulse frequency.

According to the invention it is provided that the activation modes and/or the deactivation modes are specifically predeterminable for every wavelength. This is realized, for example, through a user selection, wherein the user can activate an activation period for the relevant wavelengths for the identification of cHB and/or SaCO and/or the SaMet by at the push of a button. The activation period is then replaced by a deactivation phase after a renewed push of the button.

Alternatively it is also provided that the activation period and/or the deactivation period are automatically predeterminable for at least one wavelength. In that case, for example, the identification of cHB and/or SaCO and/or SaMet would be automatically performed every 2 to 600 seconds.

According to the invention it is provided that the activation periods and the deactivation periods for at least two wavelengths occur essentially synchronously, for example, for the identification of the oxygen saturation.

While specific embodiments of the invention have been shown and described in detail to illustrate the inventive principles, it will be understood that the invention may be embodied otherwise without departing from such principles.

We claim:

1. Method for the identification of at least two substances of content of a body fluid, the method comprising arranging adjacent to a body tissue containing the body fluid at least one radiation source and a photo receiver, wherein the radiation source generates radiation of at least two different wavelengths, directing the radiation onto the body tissue, the photo receiver receiving radiation that is reflected by the body tissue and/or receiving radiation that is reduced through the body tissue, further comprising at least temporarily directing radiation of a third wavelength onto the body tissue for the identification of a hemoglobin derivate.

2. The method according to claim 1, wherein at least three separate radiation sources are used.

3. The method according to claim 1, wherein at least two radiation sources emit at essentially the same wavelength range.

4. The method according to claim 1, wherein successively at least three wavelengths and 4 LED are used.

5. The method according to claim 1, wherein successively four wavelengths and 6 LED are used.

6. A method for the identification of at least two substances of content of a body fluid, the method comprising arranging adjacent to a body tissue containing the body fluid at least one radiation source and a photo receiver, wherein radiation of two different wavelengths is generated by the radiation source, wherein the radiation is directed onto the body tissue and the photo receiver receives radiation reflected by the body tissue or reduced through the body tissue, wherein through control of a user selection and/or through automatic control at least temporarily radiation of a third wavelength is directed onto the body tissue for the identification of the hemoglobin concentration, further comprising directing additionally at least temporarily radiation of a fourth wavelength onto the body tissue for the identification of the carbon monoxide concentration of the hemoglobin.

7. A method for the identification of at least two substances of content of a body fluid, the method comprising arranging adjacent to a body tissue containing the body fluid at least one radiation source and a photo receiver, wherein radiation of two different wavelengths is generated by the radiation source, wherein the radiation is directed onto the body tissue and the photo receiver receives radiation reflected by the body tissue or reduced through the body tissue, wherein through control of a user selection and/or through automatic control at least temporarily radiation of a third wavelength at which water absorbs more strongly than hemoglobin is directed onto the body tissue for the identification of the hemoglobin concentration, further comprising directing additionally at least temporarily radiation of a fourth wavelength in the range of 600 nm to 710 nm onto the body tissue for the identification of a carbon monoxide concentration of the hemoglobin.

8. The method according to claim 1, wherein at least two radiation sources emit an essentially equal wavelength and the intensity of the one wavelength range is increased by simultaneous use of both radiation sources.

9. A device for the execution of the method according to claim 1 for the identification of at least two substances of content of a body fluid which has at least one radiation source for the generation of radiation of two wavelengths, wherein the radiation source and the photo receiver are equipped with a clamping arrangement for positioning in the area of a body tissue containing a body fluid, and wherein the radiation source emits at least temporarily radiation of a third wavelength.

10. The device according to claim 1, wherein the radiation sources are constructed. as radiation emitting diodes (LED).

11. The device according to claim 10, wherein a plurality of LED with different wavelengths is arranged in a common housing.

12. The device according to claim 10, wherein the light emitting diodes can be activated separately.

13. The device according to claim 10, wherein at least two LED emit in the red spectrum range.

14. The device according to claim 10, wherein at least two LED emit in the infrared spectrum range.

15. The device according to claim 10, wherein at least one LED emits in the range of a wavelength at which high water absorption is present.

16. The device according to claim 10, wherein two LED emit with a given spectrum for the identification of the oxygen saturation of hemoglobin, and wherein at least temporarily at least one auxiliary LED emits certain spectral portions for the identification of the carbon monoxide saturation of hemoglobin.

17. The device according to claim 10, wherein a main LED emits in a predetermined spectrum, wherein at least one auxiliary LED complements temporarily certain intensities in the area of the spectrum of the main LED.

18. The device according to claim 10, wherein two LED emit at a predetermined spectrum for the identification of the oxygen saturation of hemoglobin, and wherein at least at times an auxiliary LED emits certain spectral portions for the identification of the hemoglobin concentration.

19. The device according to claim 10, wherein two LED emit at a predetermined spectrum for the identification of the oxygen saturation of hemoglobin, and wherein at least at times an auxiliary LED emits certain spectral portions for the identification of the methemoglobin concentration.

20. A method for identifying at least two substances of content of a body fluid, comprising arranging adjacent to a body tissue containing the body fluid at least one radiation source and a photo receiver, wherein the radiation source generates radiation of at least three different wavelengths and the radiation is directed onto the body tissue, wherein the photo receiver receives radiation reflected by the body tissue and/or reduced through the body tissue, wherein selected activation periods and selected deactivation periods are provided for at least one wavelength.

21. The method according to claim 20, wherein, during an activation period, radiation of at least three different wavelengths is directed onto the body tissue, wherein at least one of the at least three wavelengths is present only at times in all n-cycles in the activation period.

22. The method according to claim 20, wherein for the cycles N=2 is valid indefinitely.

23. The method according to claim 21, wherein at least one wavelength has an activation period different from the two other wavelengths.

24. The method according to claim 20, wherein at least for one wavelength the activation period and the deactivation period alternate.

25. The method according to claim 20, wherein at least for one wavelength the activation period and the deactivation period alternate n-cycles.

26. The method according to claim 21, wherein for the cycles N=2 applies indefinitely.

27. The method according to claim 20, wherein for at least one wavelength the deactivation period for one wavelength can be in the time range of three seconds up to one hour.

28. The method according to claim 20, wherein at least for one wavelength the deactivation period of a wavelength can last up to one hour, while at least two further wavelengths are at least at times in an activation period.

29. The method according to claim 20, wherein the activation period for at least one wavelength can last up to one hour.

30. The method according to claim 20, wherein at least for one wavelength the activation period can last up to one hour, while at least two further wavelengths are included at least temporarily in one activation period.

31. The method according to claim 20, wherein the activation periods and the deactivation periods are specifiable for each wavelength.

32. The method according to claim 20, wherein the activation periods and the deactivation periods for at least two wavelengths occur essentially simultaneously.

33. The method according to claim 20, wherein the activation period and/or the deactivation period can be specified by a user for at least one wavelength.

34. The method according to claim 20, wherein for at least one wavelength an activation mode is interrupted by a deactivation mode in order to save operating energy.
